(19) United States
(12) Patent Application Publication
(10) Pub. No.: US 2023/0420097 A1
(43) Pub. Date: Dec. 28, 2023

(54) LIFEMAP SYSTEM, ADAPTIVE LIFEMAP SYSTEM, LIFEMAP PLANNING PROCESSES, AND METHODS OF USE

(71) Applicants: Ernest Liang, South Orange, NJ (US); Leelila Strogov, South Orange, NJ (US); Johnson Song, McLean, VA (US)

(72) Inventors: Ernest Liang, South Orange, NJ (US); Leelila Strogov, South Orange, NJ (US); Johnson Song, McLean, VA (US)

(21) Appl. No.: 18/214,048

(22) Filed: Jun. 26, 2023

Related U.S. Application Data
(60) Provisional application No. 63/356,059, filed on Jun. 28, 2022.

(51) Int. Cl. G16H 20/00; G16H 50/20; G16H 10/20

(52) U.S. Cl. CPC G16H 20/00; G16H 50/20; G16H 10/20

(57) ABSTRACT

A lifemap system, adaptive lifemap system, lifemap planning processes, and methods of use is presented. Dynamic life map system is designed and configured to provide a personalized “lifemap” which serves as an adaptive guide. The present dynamic life map system provides motivation features which motivate and/or enhance a user to achieve life goals (small or large goals) or “goals”. Furthermore, in the arrangement shown as one example, the dynamic life map system provides resources which help users achieve the goals laid out for a user; resources which help a user achieve personalized goals and/or achievements.

### CROSS-REFERENCE TO RELATED APPLICATIONS

The present application claims priority to the U.S. Provisional Patent Application No. 63/356,059 which was filed on Jun. 28, 2022, which is hereby incorporated by reference herein in its entirety, including any figures, tables, or drawings.

### FIELD OF THE DISCLOSURE

This disclosure relates to a lifemap system, adaptive lifemap system, lifemap planning processes, and methods of use. More specifically, and without limitation, the present disclosure relates to an adaptive system which identifies goals, necessary achievements, and motivates an individual to achieve those goals. More specifically, and without limitation, the present disclosure relates to an adaptive system for helping an individual or group achieve goals, often associated with life planning, such as career planning, and the like.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document. Copyright. AtomicMind LLC. All rights reserved.

### BACKGROUND OF THE DISCLOSURE

In recent times, planning a career can be stressful. Furthermore, changing careers in a constantly changing employment environment can be difficult. Individuals and even organizations and groups of all sizes, ages, and other backgrounds are constantly working on planning goals and working towards achievements. Particularly, and as one example, individuals from infants (typically planned for them) and adults after a career may have goals, such as career goals, which they desire to achieve.

In career planning, as one example, the first exposure for young people to planning a career might be the question “what do you want to be when you grow up?”. This is typically a one word answer. How to get there is another, oftentimes complex matter, without any answer. If you want to be a musician and don't know any musicians, how will you find out how to be such? Similarly, how does one go about becoming a firefighter, an astronaut, or a patent examiner?

The second exposure, if a young person is lucky, to career planning is by conversations with a career counselor, guidance counselor, or perhaps teacher or instructor. This is oftentimes in a school setting, such as elementary or high school. These types of guidance counselors may have experience limited to counseling, or teaching and may not understand some of the intricacies of different careers or how to achieve the goals of those particular careers.

Choosing a career may be the most important decision of one's life. This is not an easy decision. Oftentimes particular skill sets and backgrounds are needed. Maybe even early planning from infancy could be needed for some careers or goal achievements. Some generic tests exist to help with personality tests and the like. However, the efficacy of these tests is widely debated and still do not end in providing much more than a one word answer about vague career choices.

Complicating matters further, many who choose a career may change their mind prior to finishing school, certificates needed for that career, or even in the middle of a previous career or job choice. This means that some may change education paths, wasting valuable college credits, years of time, or years invested in a career and experience gained. This can be harmful for individuals and companies, among others.

Thus, there is a long-felt need in the art for an effective and adaptive planning system which can provide information about how to achieve one's goals and also help to achieve those goals.

The disclosure herein provides these advantages and others as will become clear from the specification and claims provided.

### SUMMARY OF THE DISCLOSURE

A lifemap system, adaptive lifemap system, lifemap planning processes, and methods of use is presented. More specifically, and without limitation, the present disclosure relates to an adaptive system which identifies goals, necessary achievements, and motivates an individual to achieve those goals. More specifically, and without limitation, the present disclosure relates to an adaptive system for helping an individual or group achieve goals, often associated with life planning, such as career planning, and the like.

The lifemap system, adaptive lifemap system, lifemap planning processes, and methods of use provides the state of the art with a personalized “lifemap” which serves as an adaptive guide, motivator, and resource to help people take steps needed to achieve their life's goals; whether career related or otherwise.

Thus, it is a primary object of the disclosure to provide a lifemap system, adaptive lifemap system, lifemap planning processes, and methods of use that improves upon life planning, and particularly, provides personalized life planning and guides for a person to achieve their goals.

Another object of the disclosure is to provide a lifemap system, adaptive lifemap system, lifemap planning processes, and methods of use that accepts various types of inputs for goals from names to professions, and more.

Yet another object of the disclosure is to provide a lifemap system, adaptive lifemap system, lifemap planning processes, and methods of use that can provide names of those who might exemplify the kinds of accomplishments, lead the types of lives, and provide a route for experiences the user wishes to have.

Another object of the disclosure is to provide a lifemap system, adaptive lifemap system, lifemap planning processes, and methods of use that gathers various background information on a user.

Yet another object of the disclosure is to provide a lifemap system, adaptive lifemap system, lifemap planning processes, and methods of use that utilizes machine learning to accept and organize background information.

Another object of the disclosure is to provide a lifemap system, adaptive lifemap system, lifemap planning processes, and methods of use that provides starting points for a user based on background information such as, but not limited to, age, gender, location, ethnicity, family background, educational background, job history, personal network, professional network, and more.

Yet another object of the disclosure is to provide a lifemap system, adaptive lifemap system, lifemap planning processes, and methods of use that provides additional information to a profile based on background information, not provided by a user, to further enable the drive toward goals, and layout particular paths or path options that will lead toward the end goal.

Another object of the disclosure is to provide a lifemap system, adaptive lifemap system, lifemap planning processes, and methods of use that can create a goal path and goals based simply on the name of a person.

Yet another object of the disclosure is to provide a lifemap system, adaptive lifemap system, lifemap planning processes, and methods of use that can create a goal path and goals to achieve an end goal based on an abstract thought, such as, but not limited to “achieving happiness”, or “achieving contentedness”, or “achieving relaxation”.

Another object of the disclosure is to provide a lifemap system, adaptive lifemap system, lifemap planning processes, and methods of use that provides the ability for a user to select from a number of people who exemplify the lives and/or goals which they want to achieve and/or exemplify.

Yet another object of the disclosure is to provide a lifemap system, adaptive lifemap system, lifemap planning processes, and methods of use that provides a series of questions that utilize a predetermined set of rules and/or machine learning techniques to further personalize and enhance paths and goals and provide means and motivations for achieving those goals.

Another object of the disclosure is to provide a lifemap system, adaptive lifemap system, lifemap planning processes, and methods of use that reverse engineers a plurality of paths and/or a plurality of path options which a user can take and/or utilize to achieve and/or accomplish their end goal and/or plurality of end goals.

Yet another object of the disclosure is to provide a lifemap system, adaptive lifemap system, lifemap planning processes, and methods of use that provides the ability to customize paths and/or change paths to achieve the same end goal.

Another object of the disclosure is to provide a lifemap system, adaptive lifemap system, lifemap planning processes, and methods of use that provides assessments which enhance input and provide metrics for ability and feedback for a user.

Yet another object of the disclosure is to provide a lifemap system, adaptive lifemap system, lifemap planning processes, and methods of use that provides a dynamic life map which can adjust based on courses taken and update regularly based on step by step accomplishments and/or course corrections; these might include unexpected changes such as life changes, illness, location, and other changes.

Another object of the disclosure is to provide a lifemap system, adaptive lifemap system, lifemap planning processes, and methods of use that provide probability and progress scores.

Yet another object of the disclosure is to provide a lifemap system, adaptive lifemap system, lifemap planning processes, and methods of use that provides analytics and non-obvious tactics, techniques and strategies.

Another object of the disclosure is to provide a lifemap system, adaptive lifemap system, lifemap planning processes, and methods of use that provides resources; where resources may be associated with a particular step, a particular time, a particular location, or end goal, or the like.

Yet another object of the disclosure is to provide a lifemap system, adaptive lifemap system, lifemap planning processes, and methods of use that provide suggestions to help a user succeed along the way; whether a color of dress to wear or a suggestion on best location to live for a particular career, or the like.

Another object of the disclosure is to provide a lifemap system, adaptive lifemap system, lifemap planning processes, and methods of use that assign lifetime values to each path and/or step and the like.

Yet another object of the disclosure is to provide a lifemap system, adaptive lifemap system, lifemap planning processes, and methods of use that provides lifetime earnings expectancies which are customized to a user and goals.

Another object of the disclosure is to provide a lifemap system, adaptive lifemap system, lifemap planning processes, and methods of use that provides investment recommendations based on life path and personalized information and goals, and/or user profile.

Yet another object of the disclosure is to provide a lifemap system, adaptive lifemap system, lifemap planning processes, and methods of use that provides for investment of investors in a user.

Another object of the disclosure is to provide a lifemap system, adaptive lifemap system, lifemap planning processes, and methods of use that provides for live access to specialists; whether a career guidance specialist or a person engaging in the career desired by the user.

Yet another object of the disclosure is to provide a lifemap system, adaptive lifemap system, lifemap planning processes, and methods of use that provides the ability to zoom in and/or zoom out of a life path and/or the like to see large life goals such as retirement and/or minute goals such as picking out shoes or a dress or immediate and/or small steps in achieving overall goals and guidance.

Another object of the disclosure is to provide a lifemap system, adaptive lifemap system, lifemap planning processes, and methods of use that provides a community and social aspect so a user can engage with and facilitate relationship building.

Yet another object of the disclosure is to provide a lifemap system, adaptive lifemap system, lifemap planning processes, and methods of use that can run autonomously through machine learning and a predetermined set of rules.

Another object of the disclosure is to provide a lifemap system, adaptive lifemap system, lifemap planning processes, and methods of use that are easy to use.

These and other objects, features, or advantages of the present disclosure will become apparent from the specification and claims.

### BRIEF DESCRIPTION OF THE DRAWINGS

The drawings accompanying and forming part of this specification are included to depict certain aspects of the disclosure.

FIG. 1 is a diagram illustrating major components of the system; the diagram showing a system, a graphical user interface, an adaptive guide, a user, and a computer platform.

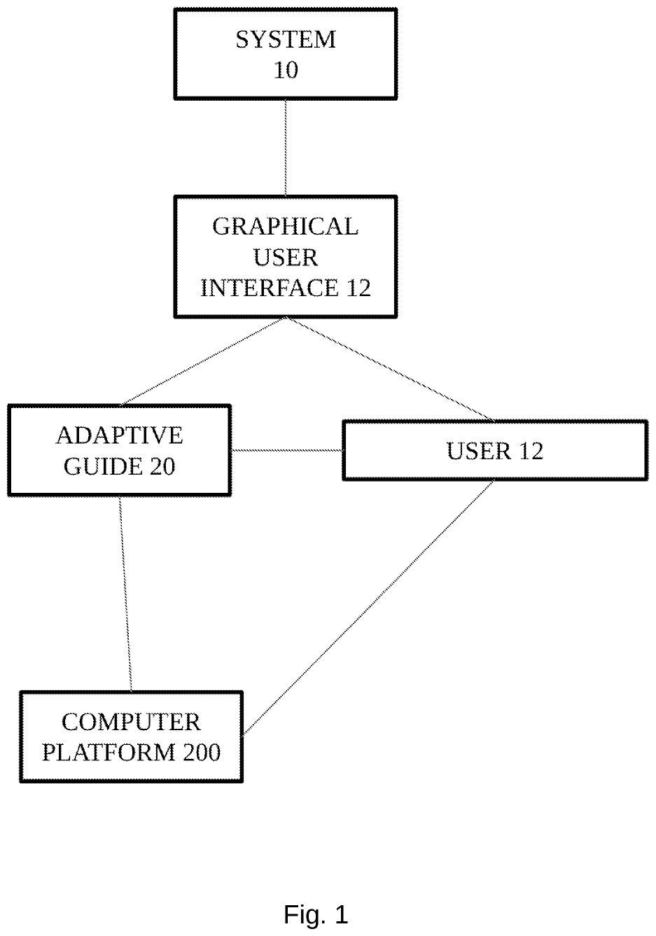

FIG. 2 is a diagram illustrating a process for creating a lifemap.

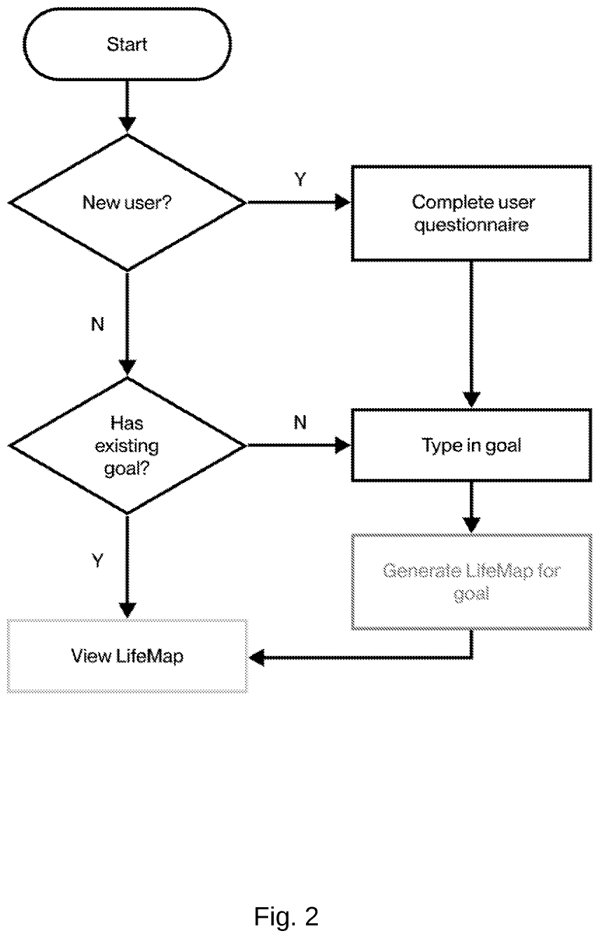

FIG. 3 is a diagram illustrating a lifemap view and various features associated with an overview of a lifemap.

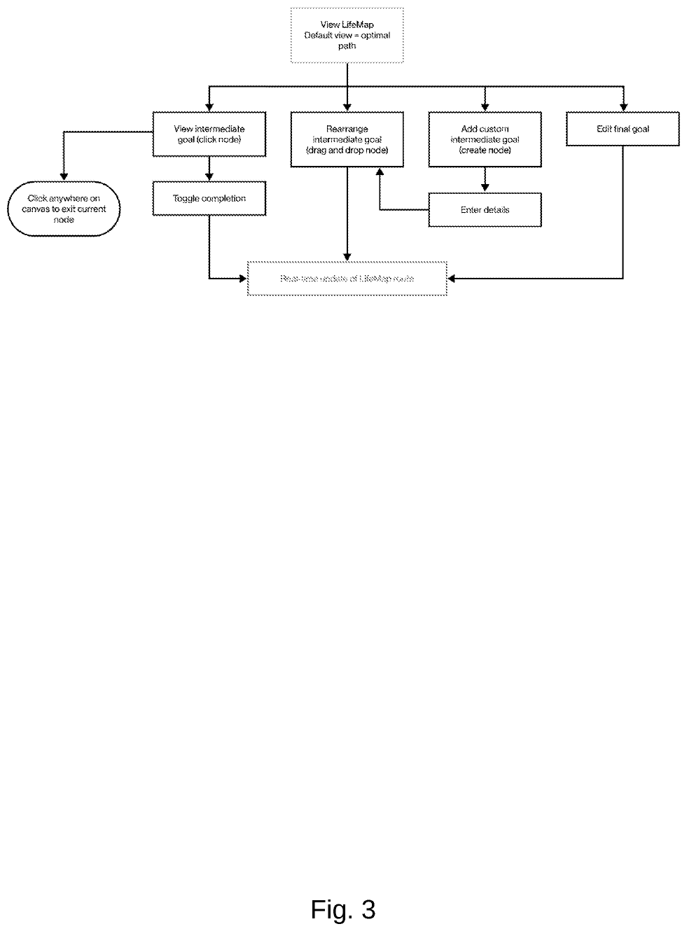

FIG. 4 is a diagram illustrating a method for creating a lifemap using the system disclosed herein.

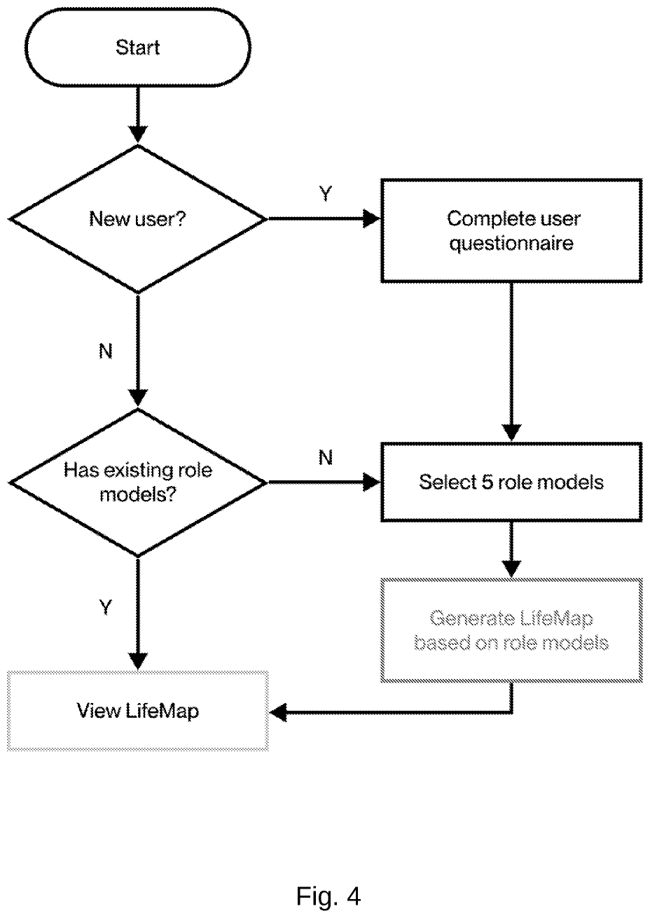

FIG. 5 is a diagram illustrating a lifemap view and various features associated with an overview of a lifemap.

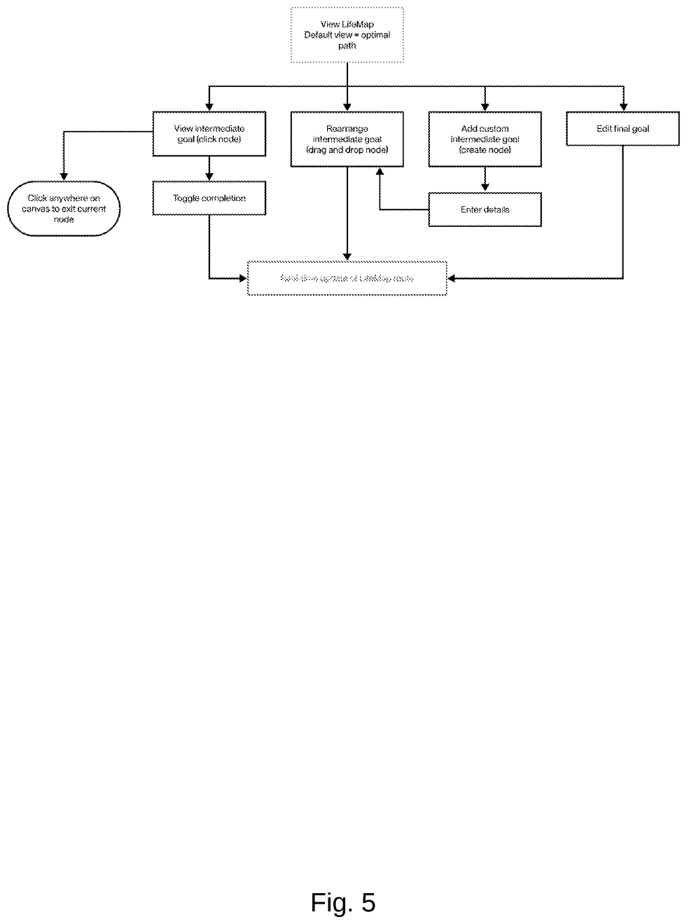

FIG. 6 is a diagram showing the system of a system and various features and functionality of the system.

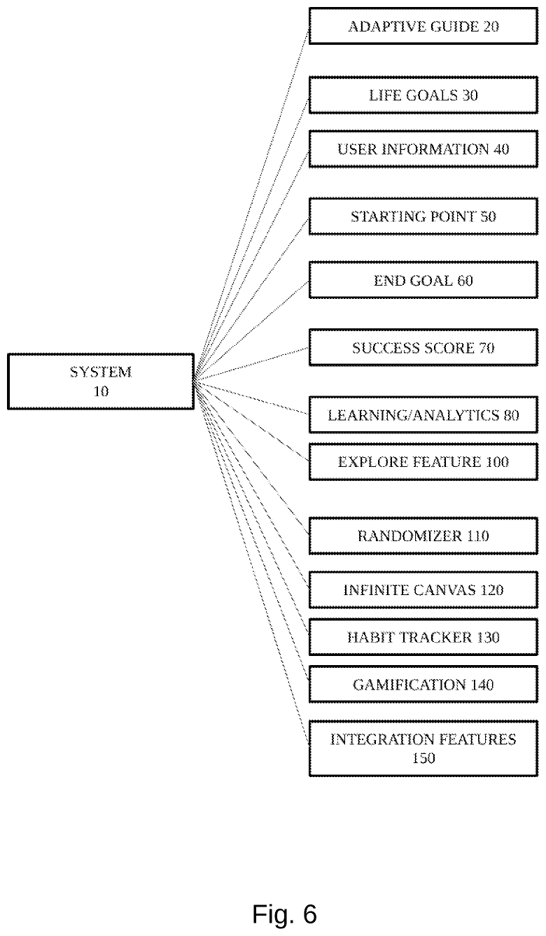

FIG. 7 is a diagram showing the system of a system and various features and functionality of the system.

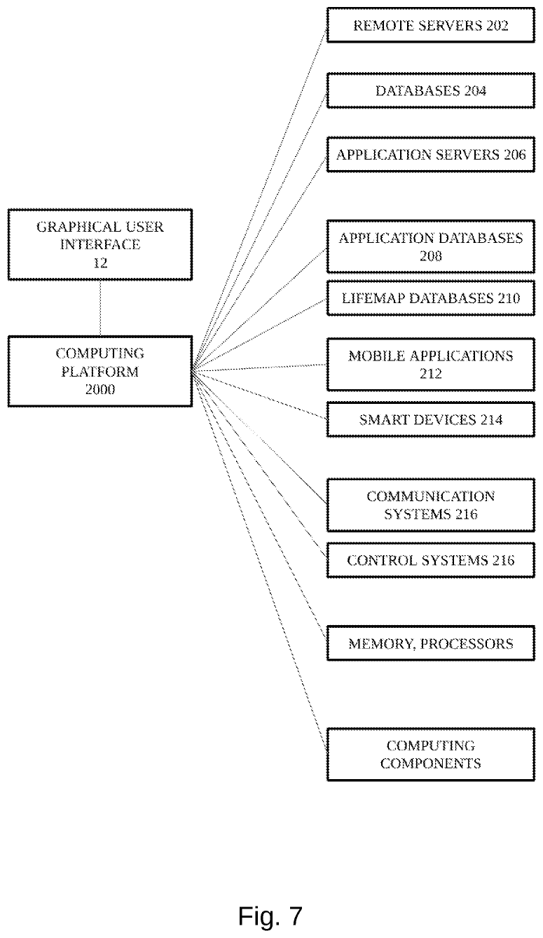

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer removable drive, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code, or virtual code, or framework code suitable for the disclosure herein, or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

In the arrangement shown, as one example, a lifemap system, adaptive lifemap system, lifemap planning processes, and methods of use is presented. More specifically, and without limitation, the present disclosure relates to an adaptive system which identifies goals, necessary achievements, and motivates an individual to achieve those goals. More specifically, and without limitation, the present disclosure relates to an adaptive system for helping an individual or group achieve goals, often associated with life planning, such as career planning, and the like.

System:

With reference to the figures and the specification herein, a lifemap system, adaptive lifemap system, lifemap planning processes, and methods of use is presented. Life map adaptive system 10 (hereafter referred to as “lifemap system”, “adaptive lifemap system”, “lifemap planning processes”, “goal planning process”, “dynamic life map system”, or simply “system”) is formed of various appearances, functionality, and personalization.

In the arrangement shown, as one example, adaptive system 10 may comprise remote servers 202, databases 204, application servers 206, application databases 208, lifemap databases 210, mobile applications 212, and/or computers or smart devices 214; all of which in continuity or as separate acts fulfill the functions disclosed herein, including but not limited to communication and control components 216, among other components, features, and functionality.

In the arrangement shown, as one example, dynamic life map system is designed and configured to provide a personalized “lifemap” which serves as an adaptive guide 20 for a user. Additionally, dynamic life map system provides motivation features which motivate and/or enhance a user to achieve life goals 30 (small or large goals) or “goals” 30. Furthermore, in the arrangement shown as one example, the dynamic life map system provides resources which help users achieve the goals laid out for a user; resources which help a user achieve personalized goals and/or achievements 30.

In one example, and as used as one example to explain an exemplary embodiment of the disclosure, life map system provides a process for achieving goals. As one example, this goal may be a career goal. In this example, the process begins with a user describing, in a single word or in great detail, the goal and/or career goal which they wish to achieve.

Some users may have specific goals, such as wanting to be an engineer, wanting to become an artist or musician, wanting to become an accountant or teacher, and similar goals. Perhaps a user's career goals might be a little more vague such as wanting to land on mars or win a Nobel Prize or become a real estate magnate. Other goals may be less outlined such as wanting to become famous, wanting to become a singer-songwriter, or wanting to provide healthcare for others or volunteer and create other societal goals and/or improvements. Some goals may be person specific like wanting to be like Taylor Swift or live a life like Egan Bernal or Lance Armstrong, or be the next Michael Jordan or Michelle Obama. Other goals may be experiential such as wanting to attend Coachella or ride in a bike race, or run a half marathon. So, the system will start with a user entering their goals, whether vague or specific, detailed or undetailed.

In the example, as being discussed, the next steps will also be entering user information 40 to help gather and determine the starting point of a user. Where is the user currently located? How old is the user? These and other various information will be determined by the system in order to understand a starting point 50. The system can learn the starting point in a variety of ways, whether it is by accessing a linkedin profile or reading the resume and/or CV of a user and/or other learning means. Some of this starting point information and background information includes, but is not limited to age, gender, zip code of location and/or city and/or state and/or country, ethnicity, place the user was born, other places the user has lived or enjoyed visiting, family background, educational background, job history, personal network of friends, personal leisure activities, professional network, and the like.

Once the system determines the end goal and/or goal post 60, the system will “reverse engineer” and/or chart a life map to help the user get from a starting point to the end goal. This can be likened to navigating from point A to point B. Similarly to navigating a physical path in the real world, various options are available to get from a starting point to achieving an end goal. The system will provide a variety of path options (if available and/or applicable), then help the user in achieving that end goal.

Similarly, the life map system allows a user to zoom in and/or zoom out of the goal path. Similarly to zooming in on a geographical navigation map to a particular turn or street view, the user can zoom in on a goal path such as a career path to a particular goal such as what color to wear to a job interview. A user can also zoom all the way out to a career goal and/or retirement age and the like.

These goals can also be modified and actively adapt to the needs of a user. For example, if a user relocates and/or moves to another city or state then the goals will be modified, the resources and human networks and professional organizations may also be modified, and the like in order to still help a user achieve their goals. A user can actively make changes to their goals as well. If a user has a goal to retire at the age of 65 but then wants to work longer, the user can adjust to this goal to a later time, and the like.

In this example, the system will utilize public information and other information to prepare these goals and may learn from other users in the system who have achieved these goals and the like. For example, if a state requires licensure to practice law, and a user wants to be an attorney, then passing the bar and other requirements to be able to take the bar in that state and the like will become part of the path for a user.

The system includes various features that help a user set, motivate, and achieve the goal and/or goals. Some of these various features include a plurality of applicable assessments. Some of these assessments might include Myers-Briggs, mental health assessments, DNA tests, and the like. Additionally, the life map system is adaptive. In this way, when changes happen in the life of a user, perhaps even achieving small goals, these goals get marked off. Depending on the timing, etc of achievements, this may change later goals, such as starting a course in the spring instead of in the fall, which will change all subsequent goals as well. Some changes may not be planned, such as unexpected illnesses, change in the desire of the goals, a change of the goal path, etc.

In the arrangement shown, as one example, the life map system will also generate a probability of success score 70 at each stage or turn of the process. These scores will help a user determine how they are doing at each stage and/or an overall score of achievement. This also helps motivate a user to achieve goals with a higher degree of success and the like.

Not all strategies for achieving a goal and/or achieving success 80 are obvious. For this reason, the present disclosure provides various resources and suggestions, many of which are hidden tactics 80 and/or strategies 80 that help. The system utilizes data analytics and machine learning to provide and enhance these strategies and tactics for a user. Some of these items may be going to a certain school to get a job with a certain employer and the like. One example of a hidden tactic may be a school to attend; this may be based on how many employees the employer has from a particular school. In this way, in addition to provide suggestions of resources for helping to achieve goals, the system will provide hidden tactics. As another example, this may be investing. The system may suggest investing in a particular company based on the goal of a user. Additionally, along with a particular type of investment may come a secondary company or second type of investment which enhances the strategy or goal. For example, a user might invest in a company by purchasing stock, but then also sell options on that stock as a secondary strategy or hidden tactic.

In the arrangement shown, the system also provides learning and analytics 80 which provide for lifetime calculations. This may be calculating lifetime expected earnings of a particular career. This information is customized to a user profile, such as how old the user is and how long it will take the user to achieve that working profession, expected increase in pay per year at that job, expected economy status through highs and lows of an economy and more. Similarly, a system may be able to predict possibilities in life expectancy and the like as determined through machine learning based on family history and DNA and the like. This can help a user to determine a more valuable life, whether based on monetary value or another value. Similarly, this may help a user get investors to invest in the life of a user.

In the arrangement shown, as one example, the adaptive life map system also has community and social features. In this way, the system suggests a user to engage with and/or facilitate introductions of relationships and/or recommendations on organizations a user should join to help achieve goals. These relationships and/or organizations may not only coincide with career goals, as one example, but may also coincide with interests, hobbies, values, and the like, which could enhance the life of a user.

Graphical User Interface:

In the arrangement shown, as one example, the system may include a graphical user interface 14. Graphical user interface 14 is formed of any suitable size shape and design and is configured to allow a user to view interact with, manipulate, and visually access system data and information, information related thereto, and/or view various data for various life paths and/or life goals and/or add information to system about the user, goals, or changes and/or accomplishment of goals and the like.

Graphical user interface 14 is an exemplary method by which systems of the present disclosure may operate and/or make programming changes to the operation of the system. Employing graphical user interface, enhances a user's interactions with the system in the form of awareness and knowledge of data within the system. System provides direct links to a user's history, settings, programming, profile, goals, life path, life maps, options, hidden strategies and/or tactics and other information related to a particular function and/or a particular component and/or a particular user and/or a particular operation. In this way graphical user interface provides a means for a user to make adjustments to the operation and functionality of the system.

User (or Plurality Thereof):

In the arrangement shown, as one example, the system includes a user 12. Users 12 may be any user interacting with or utilizing the system. This may include viewing, controlling, analyzing, manipulating, and/or interacting with system 10. Users are not limited to a single user but may be a plurality of users. Users are not limited to an individual person, but may also be an entity and the like.

Explore Feature:

In the arrangement shown, as one example, system 10 also includes an explore feature 100. Explore feature 100 (also known as “explore” or “explore mode”) provides suggestions and/or recommendations for a user. These suggestions and/or recommendations may be set by a predetermined set of rules. These suggestions and/or recommendations are also populated based on machine learning. In this way, the suggestions and/or recommendations of the explore feature can change from time to time based on input by the user, progress of a user, and the like. Furthermore, these suggestions and/or recommendations may also change based on speed at which a user is progressing and/or not progressing, location of a user, time of year, and many other factors.

In the arrangement shown, the explore feature includes various suggestions in a rolling carousel type of fashion. However, these recommendations and/or suggestions may also be provided as push notifications, notifications, email prompts, other notifications, a combination thereof, and the like.

In the arrangement shown, explore mode might offer professions to explore to a user and/or professions that may interest a user. A user can then explore these professions which might match with items such as making a user happy in life, successful, or provide professions which a user may excel at and the like. In one example, a profession might be firefighter, or teacher, or children's book author, a philharmonic orchestra conductor, professional athlete, and the like.

Similarly, and in the arrangement shown as one example, explore mode may also offer suggestions and/or recommendations related to goals of a user. Some goals of a user may include, but are not limited to, top musical charts with a song, create a youtube blog, climb mount Everest, type at 100 words per minute, see the northern lights, learn how to read music, and the like.

Similarly, and in the arrangement shown as one example, explore mode may also offer suggestions and/or recommendations related to role models. For example, explore feature may suggest Taylor Swift as a model for how to top the music charts. Similarly, explore mode may suggest Adam Ondra for mountain climbing, or a travel influencer for traveling goals, a combination thereof, and the like.

Randomizer:

In the arrangement shown, as one example, system 10 is very specific and calculated in its configuration and offerings. However, in one arrangement a user may select a randomizer 110 feature and/or an “I feel lucky feature” 110. This feature enables a user to randomize and/or randomly generate, by a set of rules or randomly through system generation, a life path and/or life map. This may be applied to an entire life map, or simply to small features such as goals of the user, and the like.

In this way, a user can search serendipitously and/or in an randomized fashion, opening additional possibilities and unlocking hidden interests and/or peaks of interest for a user. Furthermore, the randomizer includes a sliding scale. The sliding scale of the randomizer provides that a user can vary the differences from present life maps and/or goals to completely different or only slight variations or derivations. In this way, a user can adjust the tolerances to control deviations and/or variations of the system.

Infinite Canvas:

In the arrangement shown, as one example, system 10 also includes an infinite canvas 120. Infinite canvas 120 (or zoom in zoom out) is formed of various freedom and adjustment features. In this way, a user can manually add nodes, goals, and/or remove various nodes and the like. Said another way, a user can add, edit, or remove “nodes” or “goal posts” directly on the canvas and/or life map. In this way, a user can link nodes together like a dependency graph if there is an intended chronology. Furthermore, a user can “PLAY” and/or utilize a certain path, which transforms the node/canvas structure and/or life map into an interactive story. In this way, system 10 becomes more interactive and engaging.

Daily Tracker:

In the arrangement shown, as one example, system 10 also includes a daily tracker 130 and/or habit tracker 130 and/or habit former 130. The tracker 130 and/or habit former 130 is configured to provide habit and/or habit based goals based on a user profile. The habits integrate with calendars and provide notifications. In the arrangement shown, the habits can be auto populated and/or populated to the calendar of choice of a user. Furthermore, the tracker 130 generates user goals and/or habits based on the desires of a user. In the arrangement shown, as one example, tracker 130 integrates with push notifications to nudge users daily, monthly, yearly, etc. and check in on those goals and/or habits.

Gamification:

In the arrangement shown, as one example, system 10 includes a gamification feature 140. Gamification feature 140 includes games and/or other engaging elements and/or questionnaires which provide additional information about a user and/or a user behavior and/or a user interest to the system. In one example, questions such as who the user admires or strives to be like and/or meyer briggs scores and/or information related to this type of questioning and answers.

Integrations:

In the arrangement shown, as one example, system 10 also includes integration features 150. Integrations feature 150 or integrations 150 are configured to link various profiles and data for ease of use and information providing over a variety of interfaces. Said another way, integrations 150 provides with integration across various platforms, applications, social media apps, and provides system 10 with a fuller profile built through more information about and/or related to a user such as the users spotify, instagram, airbnb, duolingo, goodreads, and the like.

Computing Platform:

In the arrangement shown, as one example, a system includes a computing platform 200 (or “computer”, or “computer platform”). Computing platform 200 is formed of any suitable size, shape, and design and is configured to provide computing support, power, and computing processing for both onboard computing functionality as well as communication for off-board or server computing functionality. In this way, an onboard computing system, among other components and features on top of the platform.

In the arrangement shown, as one example, the system includes a computer. Computer is formed of any suitable size, shape, and design and is configured to provide for the main off-board computing processing and implementation of computer handling of data from data gathering performed. Computing platform 200 may include a memory, a processor, a cloud network and more.

In addition to the above identified features, options, controls, and components, the system may also include other features and functionalities, among other options, controls, and components.

It will be appreciated by those skilled in the art that other various modifications could be made to the system, process, and method of use without parting from the spirit and scope of this disclosure. All such modifications and changes fall within the scope of the claims and are intended to be covered thereby.

What is claimed:

1. A life mapping system, comprising:
an adaptive guide;
wherein the adaptive guide includes a life map;
wherein the life map provides a plurality of motivation features which motivate;
wherein the life map provides an overview of how a user achieves a plurality of life goals;
a plurality of resources;
wherein the plurality of resources are tools which aid the user in achieving the plurality of life goals;
a user profile;
the user profile including information about the user;
the life map having a starting point;
the life map having an end goal;
a success score;
wherein the success score provides ratings for an overall performance;
analytics;
wherein the analytics have a plurality of data;
wherein the analytics enhance the ability of the user to achieve the plurality of life goals by providing information about the plurality of data;
wherein the life map system creates a plurality of smaller goals which help the user achieve the end goal.

2. The system of claim 1, further comprising:
a computing platform;
the computing platform having a plurality of remote servers;
the computing platform having a plurality of databases;
the computing platform having a plurality of application servers;
the computing platform having a plurality of lifemap databases;
the computing platform having a mobile application;
the computing platform having a plurality of smart devices;
the computing platform having a communications system;
the computing platform having control features.

3. The system of claim 1, further comprising:
a graphical user interface;
the graphical user interface having a plurality of displays;
wherein the plurality of displays provide interactions with the life mapping system.

4. The system of claim 1, further comprising:
the user profile configured with a predetermined set of rules;
wherein the user profile adjusts the predetermined set of rules based on additional information;
wherein additional information is learned throughout the life map process as the user moves through the life map.

5. The system of claim 1, further comprising:
wherein the success score provides ratings for a plurality of small goals;
wherein the success score provides ratings for a plurality of large goals.

6. The system of claim 1, further comprising:
wherein the success score provides ratings for a plurality of small achievements;
wherein the success score provides ratings for a plurality of large achievements.

7. The system of claim 1, further comprising:
a plurality of strategies;
wherein a strategy of the plurality of strategies is provided to the user by the system in order to aid the user in accomplishing an associated goal.

8. The system of claim 1, further comprising:
machine learning;
wherein machine learning autonomously adjusts the predetermined set of rules based on additional information;
wherein additional information is implemented by machine learning throughout the life map process as the user moves through the life map;
wherein the life map of the user is updated based on additional information implemented by machine learning.

9. The system of claim 1, further comprising:
an explore feature;
the explore feature having a plurality of recommendations;
wherein the explore feature provides the plurality of recommendations;
wherein the plurality of recommendations changes from time to time based on changes to the life map or changes to information related to the user.

10. The system of claim 1, further comprising:
a randomizer;
the randomizer having a set of rules;
wherein the set of rules changes based on input and changes to a user profile;
wherein the randomizer generates suggestions of alternate life map options;
wherein the randomizer generates recommendations of alternate goals.

11. The system of claim 1, further comprising:
an infinite canvas;
the infinite canvas having an overall view;
wherein the overall view provides a view of the life map from the starting point to the end goal;
wherein the overall view provides a zoom in feature and a zoom out feature;
wherein the detail of information changes as the user zooms in or zooms out;
the infinite canvas having a change feature;
wherein the user can make changes to information and goals through the infinite canvas as the information appears when the user zooms in or zooms out using the zoom in feature and the zoom out feature;
wherein the zoom in feature increases the details appearing when the user focuses or zooms in on specific steps of the goals.

12. The system of claim 1, further comprising:
a habit tracker;
the habit tracker integrating with a plurality of calendars;
the habit tracker provides updates and tracking habits and behaviors of the user;
wherein the habit tracker provides a plurality of suggestions for improving habits.

13. The system of claim 1, further comprising:
a gamification feature;
the gamification feature provides rewards or badges for achievements;
the gamification feature provides rewards when the user provides additional information about the user profile and user desires.

14. The system of claim 1, further comprising:
a plurality of integration features;
wherein the plurality of integration features provide for integration of the life map system through various platforms.

15. A process of creating life goals and easily viewing life goals, comprising the steps:
starting a life map system;
determining whether a user is a new user or an existing user;
completing a questionnaire for the new user;
adding a goal;
generating a life map;
viewing a life map.

16. The system of claim 15, further comprising:
selecting a plurality of role models;
adjusting the life map goals based on the plurality of role models selected.

17. The system of claim 15, further comprising:
providing a plurality of nodes; wherein the plurality of nodes represent a plurality of intermediate goals;
viewing the plurality of intermediate goals;
toggling completion of the plurality of intermediate goals.

18. The system of claim 15, further comprising:
rearranging a plurality of nodes; wherein the plurality of nodes represent a plurality of intermediate goals;
viewing real-time updates to a life map route.

19. The system of claim 15, further comprising:
adding custom intermediate goals;
entering details related to the custom intermediate goals;
viewing real-time updates to a life map route based on custom intermediate goals.

20. A method of creating an easy to understand life goal, comprising the steps:
starting the life map system;
answering questions from a questionnaire;
creating a user profile;
typing in a life map goal;
generating a plurality of intermediate goals based on the life map goal;
viewing the life map generated by the life map goal and user profile;
adding information to the life map;
modifying the intermediate goals to create a new life map; the new life map sharing the same end goal as the life map goal.
